(19) United States
(12) Patent Application Publication
(10) Pub. No.: US 2019/0001061 A1
(43) Pub. Date: Jan. 3, 2019

(54) Automatic Injection Device Having An Improved Cam Path

(71) Applicant: Nemera La Verpillière S.A.S., LA VERPILLIERE (FR)

(72) Inventors: Kevin STAMP, Sheffield (GB); Pascal DUGAND, Estrablin (FR)

(21) Appl. No.: 16/063,551

(22) PCT Filed: Dec. 13, 2016
(86) PCT No.: PCT/FR2016/053368

§ 371 Date: Jun. 18, 2018

(30) Foreign Application Priority Data
Dec. 24, 2015 (FR) 1563313

(51) Int. Cl. A61M 5/20

(52) U.S. Cl. CPC A61M 5/2033; A61M 2005/2013; A61M 5/326; A61M 2005/2086; A61M 2005/206

(57) ABSTRACT

A device for injecting a liquid product including a piston control member intended to cooperate axially, under the effect of an elastic force, with a piston rod, linked kinematically to the piston in order to make same slide. The injection device further including at least one deceleration member, being kinematically linked to the piston control member, and mounted movable in a positioning control member with which it cooperates via a deceleration cam/deceleration cam path contact, one of the deceleration cam and the deceleration cam path being carried by the deceleration member and the other by the positioning control member, the deceleration cam path including an injection portion such that, when the deceleration cam passes through this injection portion, the piston control member causes the piston to slide, the injection portion of the deceleration cam path including at least two sections referred to respectively as fast and slow, with different inclinations.

### FIELD OF THE INVENTION

The present invention concerns the field of liquid product injection devices, notably pharmaceutical liquid product injection devices, notably automatic injection devices.

### BACKGROUND OF THE INVENTION

An automatic injection device is generally a medical device enabling the automatic administration of a liquid medication necessitating an injection. These devices in particular enable persons to inject themselves their dose of medication autonomously, for example persons suffering from rheumatoid arthritis, multiple sclerosis or diabetes or suffering an anaphylactic shock in the case of an allergy.

One example of an automatic injection device is described in the document U.S. Pat. No. 8,734,402. The device comprises an injection syringe that contains the liquid product to be injected and is fitted with a needle, and a syringe support. It generally suffices to press the device briefly onto the skin of the patient to trigger penetration of the needle into the skin, followed by injection of the liquid product, and then retraction of the needle into the interior of the device to prevent injuring a person with the needle.

To be more precise, the injection device is automatic, it comprises an injection spring exerting pressure on a piston rod via a control member that comes to abut against the piston rod, thanks to a polarizer contour provided at the proximal end of the piston rod. The piston rod therefore moves a piston, which injects the liquid product into the body of the patient. Then once the piston has reached the end of the body of the syringe, relative rotation of the piston rod and the control member releases the polarizer contour, deactivating the abutment of the control member against the piston rod, so that the piston rod is free to retract relative to the control member. Simultaneously with this release, a protection spring exerts pressure on the syringe body toward the proximal end of the automatic injection device, so that the injection syringe is retracted into the interior of the automatic injection device so that the injection needle is no longer accessible.

A difficulty can be encountered when the liquid product to be injected is viscous. In fact, a product of this kind requires time to diffuse into the skin. Also, when the piston rod is released at the end of the injection to cause it to retract relative to the control member, it can happen that some of the injected dose emerges from the skin. One possible solution is to provide devices containing doses of liquids to be injected larger than the dose to be injected in order for the dose actually remaining in the skin to correspond to the required dose. However, this leads to a waste of medical liquid and uncertainty as to the quantity of liquid actually dispensed.

Moreover, the injection flow rate of the product is higher at the start of the injection than at the end because the injection spring exerts a greater force at the start of the injection, when it is more compressed. Too high an injection flow rate can be painful for the patient and can in some cases degrade the injected product by shear of molecules of the product. Ideally, the injection device should inject the product at a constant flow rate.

### SUMMARY OF THE INVENTION

An object of the invention is to improve the control of the injection flow rate at least at certain stages of the injection.

To this end, the invention consists in a liquid product injection device, intended to receive an injection syringe comprising a syringe body, carrying an injection needle, and a piston mounted sliding axially in the syringe body in the distal direction when an injection is performed, the injection device comprising a piston control member intended to cooperate axially, because of the effect of an elastic force, with a piston rod, kinematically connected to the piston to cause it to slide, the injection device further comprising at least one deceleration member, this deceleration member being kinematically connected in its movement in translation along the injection axis X to the piston control member, the deceleration member being mounted movable in a positioning controlling member with which it cooperates via a contact between a deceleration cam and a deceleration cam path, the deceleration cam and the deceleration cam path being one carried by the deceleration member and the other by the positioning controlling member, the deceleration cam path comprising a so-called injection part such that when the deceleration cam travels over this injection part, the piston control member causes the piston to slide, the injection part of the deceleration cam path including at least two sections respectively termed fast and slow, with different inclinations, such that the axial speed of the piston control member, when the deceleration cam travels over the slow section, is less than the axial speed of this piston control member when the deceleration cam travels over the fast section.

Thus, it is possible to choose the rate of advance of the piston by choosing appropriate inclinations for each section of the cam paths. The greater the inclination relative to the injection axis over a section, the more the advance of the piston in the syringe body is decelerated when the cam is engaged in that section.

The invention can therefore make it possible to slow the product flow rate at the start and/or end of the injection, and thus improve the quality of the injection.

In the present description, the injection axis corresponds to the axis of the injection device defined by the axis of the injection needle. The injection axis will be denoted X. Moreover, the distal direction designates the direction farthest from the fingers of a user, i.e. closest to the skin of a patient at the time of an injection, and the proximal direction designates the opposite direction. In other words, it can be said that the distal direction and the distal sense are the direction and the sense toward the “front” of the injection device. In particular, the distal end of a component corresponds to the end adjoining the injection needle, and the proximal end corresponds to the opposite end. The injection device proposed here is configured to perform automatic injection of product, i.e. so that the movement of the piston to inject the product is controlled automatically, generally by a spring. Possibly, but not necessarily, the injection device is further configured to perform automatic insertion of the injection needle, i.e. the movement of the needle to penetrate into the body of the patient is also performed automatically, by a spring.

The injection device can further include one or more of the following features, separately or in combination:
the deceleration cam path includes at least two slow sections,
the two slow sections have different inclinations such that the axial speed of the piston control member, when the deceleration cam travels over a first slow section, is less than the axial speed of this piston control member when the deceleration cam travels over a second slow section,
the deceleration cam path includes three sections with different inclinations forming a fast section interleaved between first and second slow sections, so that the axial speed of the piston control member is successively slow, fast and slow when the deceleration cam travels over the slow, fast and slow sections,
the slow section of the injection part of the deceleration cam path coincides with a distal end or a proximal end of the injection part of this deceleration cam path, or with each of the distal and proximal ends,
the deceleration cam path includes at least two sections with different inclinations such that the angular movement of the deceleration cam traveling over them evolves successively in opposite rotation directions,
the deceleration cam is carried by the deceleration member and the deceleration cam path is carried by the positioning control member,
the deceleration member and the piston control member are one and the same,
the device comprises a needle control member intended to cooperate axially, because of the effect of an elastic force with a syringe support, kinematically connected to the syringe body
the deceleration member and the needle control member are one and the same,
the device comprises two deceleration members namely the piston control member and the needle control member.

### BRIEF DESCRIPTION OF THE DRAWINGS

The invention will be better understood on reading the appended figures, which are provided by way of nonlimiting example, in which:

FIG. 1 is a perspective view of an injection device in accordance with one embodiment of the invention,

FIG. 2 is an exploded perspective view of the injection device from FIG. 1,

FIGS. 3, 4 and 6 are respectively side perspective views of a piston control member, a retaining ring and an exterior casing of the injection device from FIG. 1,

FIG. 5 is a sectional perspective view from the distal end of the exterior casing from FIG. 6,

FIGS. 7 and 8 are respectively perspective side views of a positioning ring and a piston rod of the injection device from FIG. 1,

FIG. 9 is a view in cross section of the syringe support in which are assembled the injection syringe, the positioning ring and the piston rod of the injection device from FIG. 1,

FIG. 10 is a view in axial section of the proximal end of the syringe support with the elements that it contains when the injection device is assembled,

FIG. 11 is a side perspective view of a needle control member of the injection device from FIG. 1,

FIG. 12 is a side view of a positioning controlling member of the injection device from FIG. 1,

FIG. 13 is a perspective view partly in axial section of a plurality of assembled elements of the injection device from FIG. 1,

FIG. 14 is a perspective view of a positioning monitoring member suitable for an injection device in accordance with a second embodiment of the invention.

### DETAILED DESCRIPTION OF THE INVENTION

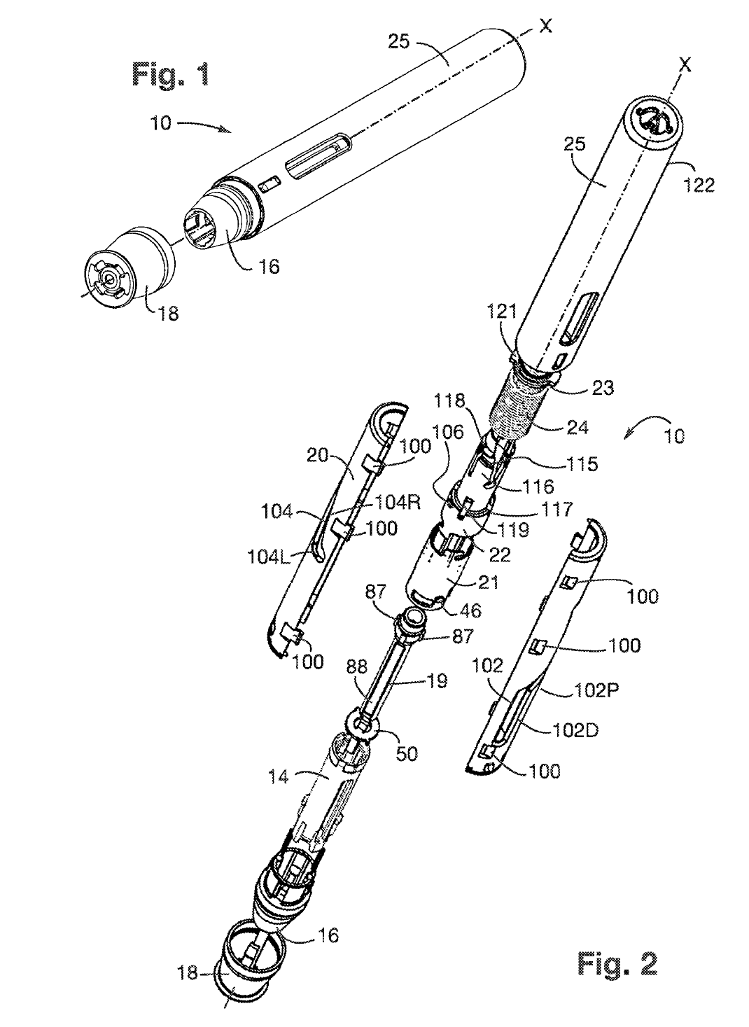

A liquid product injection device 10, as shown in FIGS. 1 and 2, enables automatic administration of a liquid product necessitating an injection, more particularly a medication. It is intended to receive an injection syringe 12 (visible in FIG. 10 in particular) containing the liquid product. In the present example, the injection device 10 is configured to perform automatic insertion of the injection needle into the body of patient and automatic injection of the liquid product. As shown in FIG. 2, the injection device 10 comprises a certain number of parts, to be more precise in this example a syringe support 14, two protection springs at the front of the injection device 10, an end sleeve 16, a member 18 for retracting a protection cap 42, a piston rod 19, a positioning ring 50, a positioning controlling member 20, a needle control member 21, a piston control member 22, a retaining ring 23, an injection spring 24, and an exterior casing 25.

As can be seen in FIG. 10 for example, the injection syringe 12 includes a syringe body 26 of globally tubular shape around an injection axis X. The syringe body 26 includes, at its distal end, an injection needle 32 (see FIG. 13). The proximal end of the syringe body 26 comprises a flange 38. A piston 40, sliding axially in the syringe body 26, of globally cylindrical shape around the injection axis X, is mounted to slide axially in the syringe body 26 and enables injection of a liquid product contained in the syringe body 26 via the injection needle 32. The piston 40 includes a distal end oriented toward the injection needle 32 and a proximal end 40P in contact with the piston rod 19. The distal end of the piston rod 19 can in particular be screwed into the piston 40.

The injection syringe 12 is, in this example, a pre-filled glass syringe, with glued-on needle, having a capacity of 1 ml (milliliter). It will be noted that the syringe body 26 delimits a maximum liquid receiving volume, but that filling it only partially can be envisaged, by advancing the piston 40 toward the distal end of the syringe body 26.

As is shown in FIGS. 10 and 13, the syringe support 14 is of tubular general shape around the injection axis X, is open at its two ends and is intended to receive the injection syringe 12. The syringe support 14 is thus a one-piece component, separate from two half-shells assembled to one another. The syringe support 14 is configured to house the injection syringe 12 so that it is fixedly mounted in the syringe support 14 throughout the operation of the injection device 10. The syringe support 14 has a distal end disposed at the same end as the injection needle 32 and an opposite proximal end 14P.

As can be seen in FIG. 10, the syringe support 14 includes, at its proximal end 14P, an internal step 44 delimited by an internal shoulder, enabling reception of the flange 38 of the syringe body 26 when the injection syringe 12 is inserted in the syringe support 14. An internal step 44 of this kind makes it possible for the proximal end 14P to have a smaller diameter than the rest of the syringe support 14 and thus to be able to receive, on the one hand the flange 38, on the other hand the positioning ring 50 of the piston rod 19, these two elements having a greater diameter than the syringe body 26. The proximal end 14P of the syringe support 14 moreover includes an external shoulder 45, disposed axially in the vicinity of the internal step 44. This external shoulder 45 forms a raised member for immobilizing the needle control member 21. To be more precise, the external shoulder 45 makes it possible to attach the needle control member 21 to the proximal end 14P of the syringe support 14 at the time of assembly of a proximal assembly with a distal assembly of the injection device 10 and until the end of the insertion of the injection needle 32 into the skin of the patient. The needle control member 21 is therefore kinematically connected to and cooperates axially with the syringe support 14 until the end of the insertion of the injection needle 32 in the skin of the patient. As can be seen in FIG. 13, the syringe support 14 enables fixing of the end sleeve 16 to the injection device 10.

The piston rod 19 is kinematically connected to the piston 40 and enables pressure to be transmitted to the piston 40 in order to inject the liquid product. The piston rod 19 comprises a distal end 19D and a proximal end 19P.

The piston rod 19 is moreover configured to be mobile between a plurality of configurations. The first configuration is a push configuration in which the piston rod 19 is able to move the piston 40 in the syringe body 26 because of the effect of the injection spring 24 between an initial position and an end of travel position. In the end of travel position, the piston 40 is at the end of the syringe body 26, i.e. toward the distal end of the syringe body 26. The second configuration is a release configuration in which the piston rod 19 can be retracted in the proximal direction of the injection device 10.

The movement from the push configuration to the release configuration is effected by rotation of the piston control member 22 relative to the piston rod 19. This rotation of the piston control member 22 and the connection with the withdrawal of the injection needle 32 will be explained below.

As shown in FIG. 8, the piston rod 19 is of globally cylindrical shape around the injection axis X. Two slide grooves 88 are provided on respective opposite sides of the piston rod 19. Each slide groove 88 can, for example, be formed by an axial groove. These slide grooves 88 extend axially (parallel to the injection axis X) along the piston rod 19 and delimit a polarizer contour (seen notably in FIG. 9). These slide grooves 88 are intended to cooperate with a passage opening 92 of the positioning ring 50. The piston rod 19 moreover comprises a distal abutment 90 for axial retention of the positioning ring 50, to be more precise two diametrically opposite distal abutments 90. Each distal abutment 90 is preferably formed by a transverse groove provided in a localized filling of the slide grooves 88. The proximal end of the piston rod 19 is of globally cylindrical shape around the injection axis X in its distal part and comprises four radial projections 87, distributed angularly around the injection axis X, intended to cooperate with the piston control member 22.

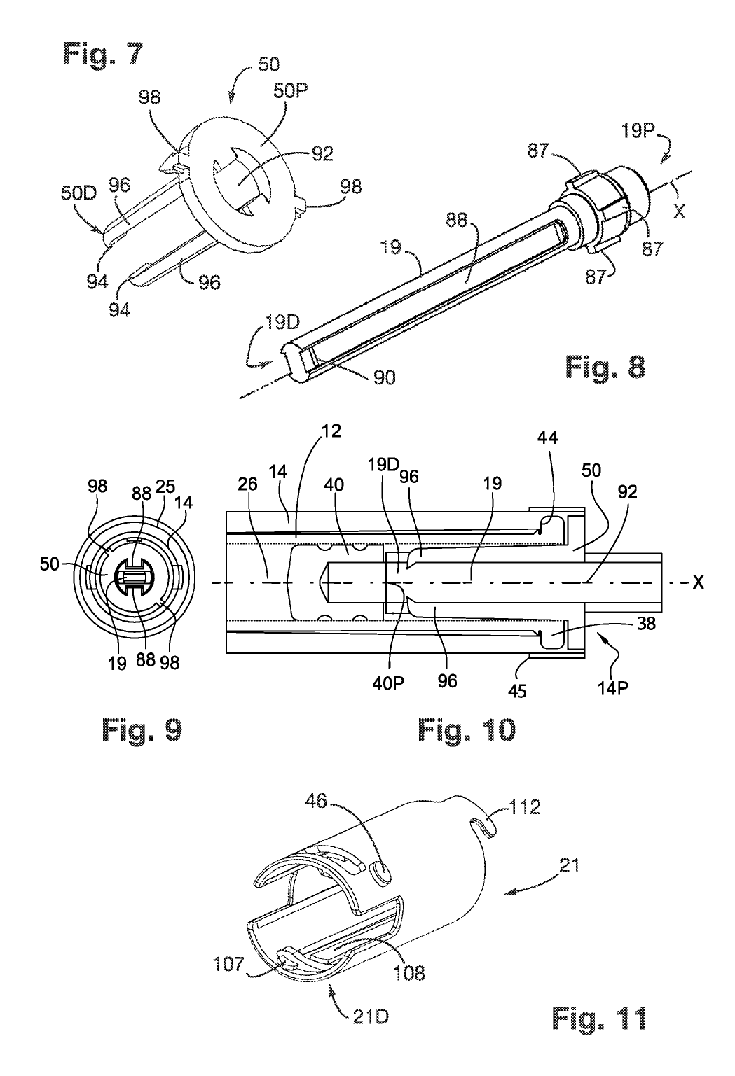

The positioning ring 50, shown in FIG. 7, is intended to cooperate with the piston rod 19 to enable, simultaneously, correct axial and angular positioning of the piston rod 19, and prevention of the withdrawal of the piston rod 19 from the operating ring 50, or even from the injection device 10. The axial positioning enables a correct position relative to the piston 40 on the one hand and to the piston control member 22 on the other hand. The angular positioning enables not only prevention of the piston rod 19 from turning on itself during activation of the piston rod 19 but also provides a predefined angular orientation relative to the syringe support 14, enabling axial abutment followed by axial release of the piston rod 19 and the piston control member 22, these configurations being implemented following rotation of the piston control member 22 relative to the piston rod 19. The positioning ring 50 is intended to be mounted at the proximal end 14P of the syringe support 14. In order to prevent free rotation of the positioning ring 50 relative to the syringe support 14, the positioning ring 50 comprises means 98 for angularly keying the positioning ring 50 to the proximal end 14P of the syringe support 14. The means for axial positioning of the piston rod 19 provided on the positioning ring 50 include an abutment 94 for axial retention of the piston rod 19, carried here by an elastic lug 96 extending axially from a proximal washer 50P toward a distal end 50D of the positioning ring 50. To be more precise (see FIG. 11), the positioning ring 50 comprises two diametrically opposite elastic lugs 96, each carrying an axial retention abutment 94, which is formed in this example by a lug for axial retention of the piston rod 19. Each axial retention abutment 94, by cooperating with the corresponding distal abutment 90 (seen only in FIG. 8) of the piston rod 19, prevents the latter from being able to be withdrawn entirely (by traction in the proximal sense) from the positioning ring 50 before assembly, and from the injection syringe 12 after assembly. The means for axial positioning of the piston rod 19 on the positioning ring 50 are moreover adapted so that, in the storage configuration of the injection device 10, a clearance is present between the distal end 19D of the piston rod 19 and the proximal end 14P of the piston 40.

The positioning ring 50 moreover includes means for angular positioning of the piston rod 19 on the positioning ring 50, comprising a passage opening 92 (visible in FIGS. 7 and 10 in particular) for the piston rod 19 having a polarizer contour complementary to that of the distal end 19D of the piston rod 19. In this way, the passage opening 92 provides defined and controlled angular positioning of the piston rod 19 relative to the positioning ring 50 throughout the operation of the injection device 10.

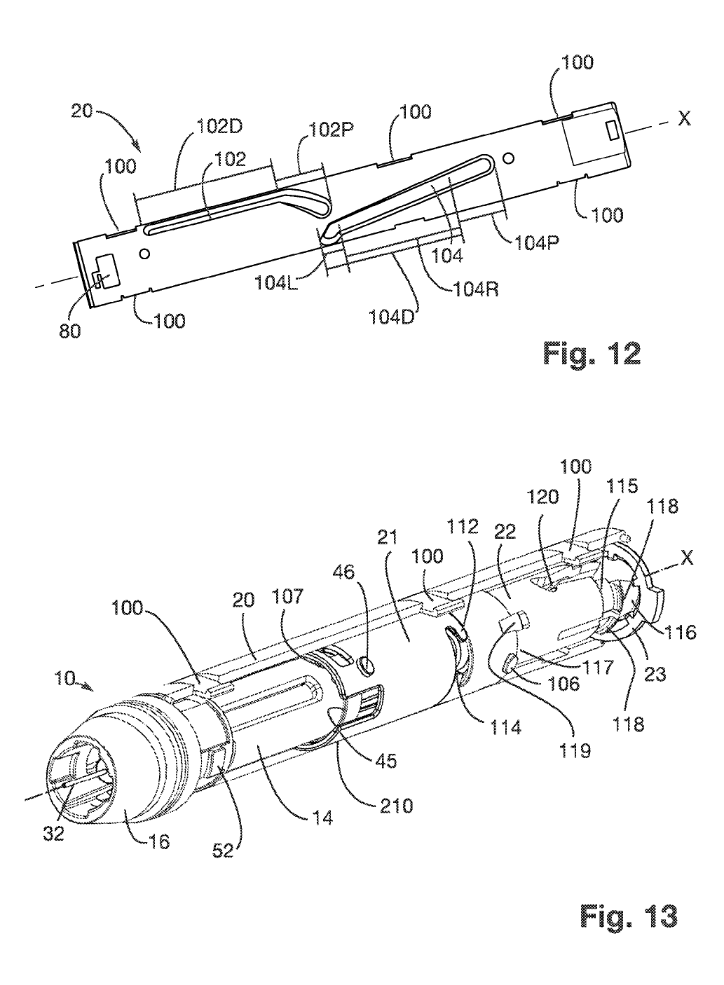

In FIG. 12 is shown the positioning controlling member 20, which comprises two parts, two half-shells intended to be clipped together around the syringe support 14 by clipping means 100 comprising various notches and lugs. The positioning controlling member 20 therefore has a tubular general shape along the injection axis X. The distal end of the positioning controlling member 20 includes means for retaining the end sleeve 16. Once assembled, the end sleeve 16 and the positioning controlling member 20 are fixed relative to one another throughout the operation of the injection device 10. As can be seen in FIG. 12, the positioning controlling member 20 also includes a needle control cam path 102 and a piston control cam path 104 intended to cooperate respectively with a needle control cam 46 and a piston control cam 106 carried by the needle control member 21 and the piston control member 22, respectively (FIG. 13). Because the needle control member 21 and the piston control member 22 are associated with respective separate cam paths, once the injection device 10 is activated, the step of insertion of the injection needle 32 into the body of the patient and the step of injection of the liquid product contained in the injection syringe 12 are therefore distinct and separate steps, so that each can be executed precisely, in a controlled time and in a manner appropriate to the injected liquid product.

According to variants, the needle control cam path and/or the piston control cam path are respectively carried by the needle control member and the piston control member, and the needle control cam and/or the piston control cam are consequently carried by the positioning controlling member.

The needle control cam path 102 and the piston control cam path 104 each comprise two parts, one distal 102D and 104D and the other proximal 102P and 104P. During the step of insertion of the injection needle 32, the needle control cam 46 and the piston control cam 106 are engaged in the proximal parts 102P and 104P of the needle control cam path 102 and the piston control cam path 104. During the step of injection of the liquid product, the needle control cam 46 and the piston control cam 106 are engaged in the distal parts 102D and 104D of the needle control cam path 102 and the piston control cam path 104. The distal parts 102D and 104D of the needle control cam path 102 and the piston control cam path 104 are termed injection parts 102D and 104D of the cam paths 102 and 104.

In this first embodiment, the piston control member 22 is a deceleration member 22. The piston control cam path 104 is therefore a deceleration cam path 104 and the piston control cam 106 is therefore a deceleration cam 106.

The injection part 104D of the deceleration cam path 104 includes two sections, one proximal, termed the fast section 104R and the other distal, termed the slow section 104L. The fast section 104R has exactly the same inclination as the proximal part 104P of the deceleration cam path 104. Merely by observing the positioning controlling member 20, it is therefore not possible to distinguish the boundary between the distal part 104D and the proximal part 104P of the deceleration cam path 104. The slow section 104L has a greater inclination relative to the injection axis X than the fast section 104R. The choice of a greater inclination over a section enables the axial speed of the deceleration member 22 to be reduced when the deceleration cam 106 travels over that section. In fact, a greater rotation of the deceleration member 22 is then necessary to cover the same axial distance. This greater rotation causes more friction in the various parts of the mechanism and therefore limits the speed of the deceleration member 22. The deceleration member 22 therefore has a lower axial speed when the deceleration cam 106 travels over the slow section 104L than when it travels over the fast section 104R. This low axial speed of the deceleration member 22 at the end of injection allows time for the product to diffuse. This makes it possible to guarantee that all the pressurized liquid has been injected before the injection needle 32 is withdrawn.

It is seen in FIG. 12 that the proximal part 102P of the needle control cam path 102 has a greater inclination relative to the injection axis X than the rest of the needle control cam path 102. This proximal part 102P is inclined at approximately 45°, which enables progressive advance of the injection needle 32, and therefore limits the patient's pain. The speed of advance of the injection needle 32 can be adjusted by modifying the inclination of the control cam path 102.

The insertion of the injection needle 32 ends when the needle control member 21 reaches a given angle. At this moment, the needle control member 21 is separated from the syringe support 14, and the injection begins. The proximal part 102P of the needle control cam path 102 causes the needle control member 21 to turn to this angular position. The slope and the length of this proximal part 102P of the needle control cam path 102 therefore makes it possible to guarantee that the injection needle 32 is inserted to the correct depth and at the correct speed into the skin of the patient before the injection begins.

According to a variant, it is the needle control member 21 that serves as the deceleration member. In this case, the needle control cam path is a deceleration cam path including an injection part comprising a slow section and a fast section, the slow section having an inclination relative to the injection axis X greater than that of the fast section so that slower injection is obtained at the start of injection and faster injection is obtained at the end of injection, for example, or vice versa.

The needle control member 21 has a tubular general shape, as can be seen in FIGS. 11 and 13. The needle control member 21 is movably mounted in the positioning controlling member 20. At its proximal end, it comprises temporary coupling means 112 with complementary coupling means 114 carried by the piston control member 22 (FIG. 5), so that the needle control member 21 and the piston control member 22 are constrained to move together at the beginning of the thrust exerted by the injection spring 24. When the injection spring 24 begins to push on the piston control member 22, it therefore also pushes on the needle control member 21 which, being at this time also coupled to the syringe support 14, will push the syringe support 14 in the distal direction, and thus enable insertion of the injection needle 32 into the skin of the patient. To be more precise, the temporary coupling means 112 and the complementary coupling means 114 include two complementary parts having a mutual attachment shape, for example two inverted hooks extending axially, the first carried by the proximal end of the needle control member 21 and the second carried by the distal end of the piston control member 22. Moreover, the needle control member 21 comprises a locking pin 107 (just visible in FIG. 13) projecting from its interior surface and having the shape of a funnel, the thin portion of the funnel being oriented on the distal side. The locking pin 107 is intended to cooperate with the external shoulder 45 of the proximal end 14P of the syringe support 14. In fact, this external shoulder 45 forms a raised portion for immobilizing the needle control member 21 at the start of operation of the injection device 10, during insertion of the injection needle 32 into the skin of the patient. As specified above, the external shoulder 45 therefore enables attachment of the needle control member 21 to the proximal end 14P of the syringe support 14 at the time of assembling the proximal end and the distal end of the injection device 10. This assembly takes place through axial insertion of the locking pin 107 in a guide rail (not shown) of the syringe support 14, followed by rotation of the needle control member 21 relative to the syringe support 14 so that, when the injection device 10 is in the storage position before being used, the external shoulder 45 is wedged axially (along the injection axis X) between the locking pin 107 on the distal side and the distal end of a guide rib (not shown) on the proximal side.

The needle control member 21 further includes, on its exterior surface and at its distal end 21D, the needle control cam 46 (see FIG. 13) intended to cooperate with the needle control cam path 102 (see FIG. 12) formed in the positioning controlling member 20.

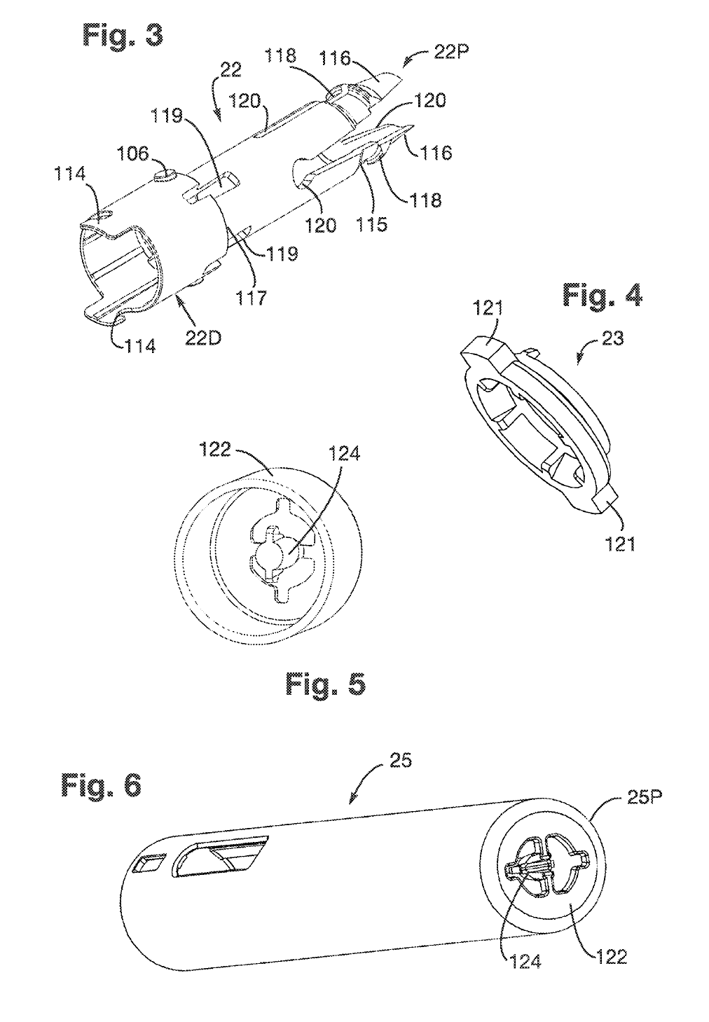

The piston control member 22, visible in particular in FIGS. 3 and 13 has a tubular general shape. It is movably mounted in the positioning controlling member 20. It is configured to apply a force to the piston 40 (via the piston rod 19), when acted on by the injection spring 24 (visible in FIG. 2). This force enables axial movement (along the injection axis X) of the piston 40 in the distal direction of the injection device 10, to inject the liquid product into the skin of the patient.

The piston control member 22 carries an abutment 118 retractable between an active configuration for retaining the injection spring 24 in the compressed state and a retracted configuration releasing the injection spring 24. To be more precise, the piston control member 22 comprises two semi-flexible, diametrically opposite, semi-annular elastic lugs 116, each carrying a retractable abutment 118 intended to cooperate with the locking member of the exterior casing 25. Each of the semi-annular elastic lugs 116 carrying the retractable abutments 118 extends axially (along the injection axis X) toward the proximal end of the injection device 10, each semi-annular elastic lug 116 therefore being deformable between a radially spaced rest configuration imparting to the retractable abutment 118 its active configuration and a radially retracted configuration imparting to the retractable abutment 118 its retracted configuration.

The exterior casing 25 comprises a member 122 for locking the retractable abutment 118 in its active configuration, provided in the proximal end of an exterior casing 25 of the injection device 10. This locking member 122 provided at the proximal end 25P of the exterior casing 25 comprises a radial surface, opened out (by means of two separate, facing orifices) in the vicinity of an unlocking pin 124 so as to have the semi-annular elastic lugs 116 of the piston control member 22 carrying the retractable abutments 118 pass through it at least when the locking member 122 is in the locking position. The locking pin 122 comprises two sections of different diameters causing the passage of the retractable abutments into the retracted position by virtue of the distal end of the injection device 10 bearing on the skin of a patient.

As can be seen in FIGS. 3 and 13, the piston control member 22 has, at its distal end, a cylindrical general shape carrying the complementary coupling means 114 and the piston control cam 106, and limiting an exterior, proximal shoulder 115 forming a seat for the injection spring 24. The piston control member 22 also includes a distal shoulder 117, this distal shoulder 117 is interrupted by notches, extending toward the proximal end of the piston control member 22 so as to form axial slots 119 (visible in FIG. 3). The piston control cam 106 carried by the piston control member 22 is intended to cooperate with the piston control cam path 104 (visible in FIG. 12) provided in the positioning controlling member 20 to regulate the movement of the elements of the injection device 10 by the effect of the injection spring 24 (see FIG. 2). The rotation of the piston control member 22 is induced by the axial movement of the piston control cam 106 in the piston control cam path 104.

As stated above, the piston rod 19 comprises a globally cylindrical part provided with at least one radial projection 87, configured to cooperate with the piston control member 22 activated by the injection spring 24.

The relative angular movement of the piston control member 22 with respect to the piston rod 19 occurs during the advance of the latter in the syringe body 26, i.e. during the injection of the liquid product. The relative angular movement of the piston control member 22 (with respect to the piston rod 19) is effected by a predetermined angle, for example close to 45°.

The second part of the angular movement considered is preferably greater than 1°, for example close to 2°. This second part of the movement of the piston control member 22 around the piston rod 19 essentially includes an angular component. In fact, the slow section 104L of the distal part 104D of the piston control cam path 104 takes account of the rotation that remains before alignment of the radial projections 87 with the axial slots 119 when the piston rod 19 has stopped being pressed into the syringe body 26. In the released configuration of the piston rod 19, the piston control member 22 is no longer abutted against the radial projections 87 of the piston rod 19 and frees a passage so that the piston rod 19 can be retracted in the proximal direction.

The retaining ring 23 makes it possible to retain the injection spring 24 in the compressed state (see FIGS. 2 and 4). To be more precise, before injection, the injection spring 24 is held in its compressed state by means of the retractable abutment 118 carried by the piston control member 22, cooperating with a complementary surface carried by the retaining ring 23 (see FIG. 13). This retaining ring 23 is attached to the proximal end 20P of the positioning controlling member 20. To this end, it includes two diametrically opposed, exterior lugs 121 for keying the retaining ring 23 intended to cooperate with complementary notches of the positioning controlling member 20. The distal end of the retaining ring 23 moreover forms a proximal seat for the injection spring 24, and the proximal end forms a complementary abutment surface for the retractable abutment 118.

The abutment 118 is retractable between an active configuration retaining the injection spring 24 in the compressed state, corresponding to a radially projecting retaining position of the injection spring 24, and a retracted configuration to release the injection spring 24 allowing it to expand. In the active configuration the retractable abutment 118 is therefore abutted against a complementary surface carried by a retaining ring 23 of the injection spring 24. This retaining ring 23 is attached to the proximal end of the positioning controlling member 20.

The injection spring 24 is the main driving force of the injection. Also, it must produce a force sufficient to be capable, on the one hand, of causing the injection needle 32 to penetrate into the body of the patient, and, on the other hand, to cause the piston 40 to move in the syringe body 26 to inject the liquid product. Its force is preferably greater than 20 Newton, or even 50 Newton or again 80 Newton for a relatively viscous liquid product or for a high syringe/needle section ratio. This injection spring 24 is deformable between a compressed state before use of the injection device 10 and a relaxed state after injection of the liquid product contained in the syringe body 26.

The exterior casing 25 (see FIGS. 1 and 2) is the component which, in this example, envelops most of the parts of the injection device 10. In particular it makes it possible to isolate these parts from the outside environment.

The main steps of the operation of the injection device 10 are described below.

As can be seen in FIG. 1, the injection device 10 is presented to the user in the form of a tube around ten centimeters long. Only the exterior casing 25 and the withdrawal member 18, or the end sleeve 16 if the withdrawal member 18 has been withdrawn, are visible and accessible to the user.

Before use of the injection device 10, the injection needle 32 is protected by a cap not shown in the figures. This cap is withdrawn with the aid of the withdrawal member 18 shown in FIG. 1. A first operating step consists in holding the withdrawal member 18 in one hand and the exterior casing 25 in the other hand and pulling on the withdrawal member 18 along the injection axis X in a distal direction in order to bare the injection needle 32. The injection needle 32 remains inaccessible, however, because, as shown in FIG. 5, it is enveloped by the end sleeve 16.

Once the withdrawal member 18 has been withdrawn from the injection device 10, the end sleeve 16 forms its distal end and the injection device 10 is ready to be used. At this stage, the injection needle 32 is retracted inside the end sleeve 16, for example by 3 mm (millimeters), to prevent accidental pricking.

According to a next step, the distal end of the end sleeve 16 is placed against the skin of the patient, at the place where the injection of the medical liquid contained in the injection syringe 12 is required.

That step is followed by an unlocking step, during which slight axial pressure is applied, in the distal direction, to the exterior casing 25. This bearing of the distal end of the injection device 10 on the skin of the patient triggers axial movement of the exterior casing 25 toward the end sleeve 16. In other words, the end sleeve 16 slides a little way inside the exterior casing 25 toward it proximal end, generating sliding, inside the exterior casing 25 and toward the proximal end of the exterior casing 25, of the positioning controlling member 20 and the retaining ring 23. This sliding results in the unlocking of the retractable abutment 118 carried by the proximal end of the piston control member 22.

The unlocking step is followed by a step of triggering automatic injection, during which the user continues to press axially on the exterior casing 25, pressing it onto the skin of the patient, in continuity with the axial pressure begun during the unlocking step. This additional pressure generates compression of the protection springs (not shown) which, firstly, enables axial movement toward each other (along the injection axis X) of the end sleeve 16 and the syringe support 14. The end sleeve 16 therefore slides further inside the exterior casing 25 toward its proximal end, generating additional sliding, inside the exterior casing 25 and toward the proximal end of the exterior casing 25, of the positioning controlling member 20 and the piston control member 22. Because of this, the retractable abutment 118 carried by the piston control member 22 slides toward the proximal end of the exterior casing 25, which generates its retraction out of the retaining ring 23, by the ramp effect between the retractable abutment 118 and the proximal surface of the exterior casing 25, so that the injection spring 24 is released.

The release of the injection spring 24 triggers a series of cascaded reactions that lead to the penetration of the injection needle 32 into the body of the patient and the injection of the liquid product contained in the syringe body 26. It will be noted that the travel of the injection needle 32, during these operations, is of 9 mm, so that the injection needle 32 projects approximately 6 mm, corresponding to the depth of injection into the skin of the patient. In particular, the injection spring 24 pushes in the distal direction on the piston control member 22 which, at this stage, is coupled to the needle control member 21 thanks to the overlapping of the temporary and complementary coupling means 112, 114. The movement of the needle control member 21, being fastened to the syringe support 14 via the external shoulder 45, generates the movement in the distal direction of the syringe support 14. As the injection syringe 12 is mounted on and fastened to the syringe support 14, the injection syringe 12 is also pushed toward the distal end of the injection device 10 and the injection needle 32 protrudes out of the end sleeve 16. This movement toward each other in the axial direction of the end sleeve 16 and the syringe support 14 is effected by sliding of the syringe support 14 in the end sleeve 16. Once the injection needle 12 is inserted in the skin of the patient, the injection can begin. It will be noted that simultaneously with the penetration of the injection needle 32, the needle control cam 46 of the needle control member 21 is guided by the corresponding needle control cam path 102 of the positioning controlling member 20, to be more precise by the part inclined at 45° of that needle control cam path 102. The result of this inclination at 45° is braking of the expansion of the injection spring 24, such that the speed of penetration of the injection needle 32 into the skin of the patient is lower than the unbraked speed of expansion of the injection spring 24, which advantageously reduces the pain linked to the penetration of the injection needle 12.

Simultaneously with the axial movement of the needle control member 21, rotation of the piston control member 22 relative to the positioning controlling member 20 is generated about the injection axis X, because the piston control cam 106 cooperates with the inclined piston control cam path 104. The result of this rotation is uncoupling of the needle control member 21 and the piston control member 22, together with coupling of the piston control member 22 and the piston rod 19, effected by the piston control member 22 abutting on the piston rod 19, to be more precise on the radial projections 87 of the piston rod 19 (see FIG. 9). The thrust exerted by the injection spring 24 on the piston control member 22 results in a thrust on the piston rod 19, and therefore on the piston 40 in the syringe body 26, implying injection of the liquid product into the body of the patient.

Clearly the relative axial advance of the needle control member 21 and the piston control member 22 is monitored by the cooperation of the needle control cam 46 and the piston control cam 106 with the corresponding needle control cam path 102 and piston control cam path 104 of the positioning controlling member 20. The cooperation between the needle control cam 46, the piston control cam 106 of the needle control member 21 and the piston control member 22 and the corresponding needle control cam path 102 and the piston control cam path 104 of the positioning controlling member 20 enables independent control of penetration of the injection needle 32 into the skin of the patient and injection of the medical liquid contained in the syringe body 26. It will be noted that the mechanism of successive coupling, comprising coupling in a first stage of the injection needle control member 21 and the piston control member 22, and coupling in a second stage of the piston control member 22 and the piston rod 19, enables total independence of the penetration of the injection needle 32 and the injection, i.e. the correct depth of the injection needle 32 is bound to be reached before the injection begins, which prevents incorrect injection.

Once the injection is finished, the piston control member 22 completes its rotation about the piston rod 19 and the piston rod 19 is again in the release configuration. The injection spring 24 has expanded and the two protection springs (not shown) can expand freely: in fact, they are no longer subjected to the force exerted by the injection spring 24 and therefore generate a protection position in which the syringe support 14 is axially spaced from the end sleeve 16. This axial spacing results in the retraction of the injection needle 32 into the end sleeve 16, thus preventing any risk of injury. At the end of the injection, the syringe support 14 therefore rises freely in the piston control member 22. Because of the rotation of the latter that made it possible to align the radial projections 87 of the piston rod 19 with the axial slots 119, a passage has been freed so that the piston rod 19 can be retracted relative to the piston control member 22, in the proximal direction and not impede the retraction of the syringe body 26 entrained by the syringe support 14.

The syringe support 14 is therefore axially spaced from the end sleeve 16, and the injection needle 32 is retracted into the end sleeve 16, thus preventing all risk of injury. As the piston rod 19 and the piston control member 22 are unfastened from one another (because the axial slots 119 are cooperating with the radial projections 87), the syringe body 26, entrained by the syringe support 14, rises in the injection device 10 and entrains the injection needle 32 in its movement. The injection needle 32 is therefore retracted.

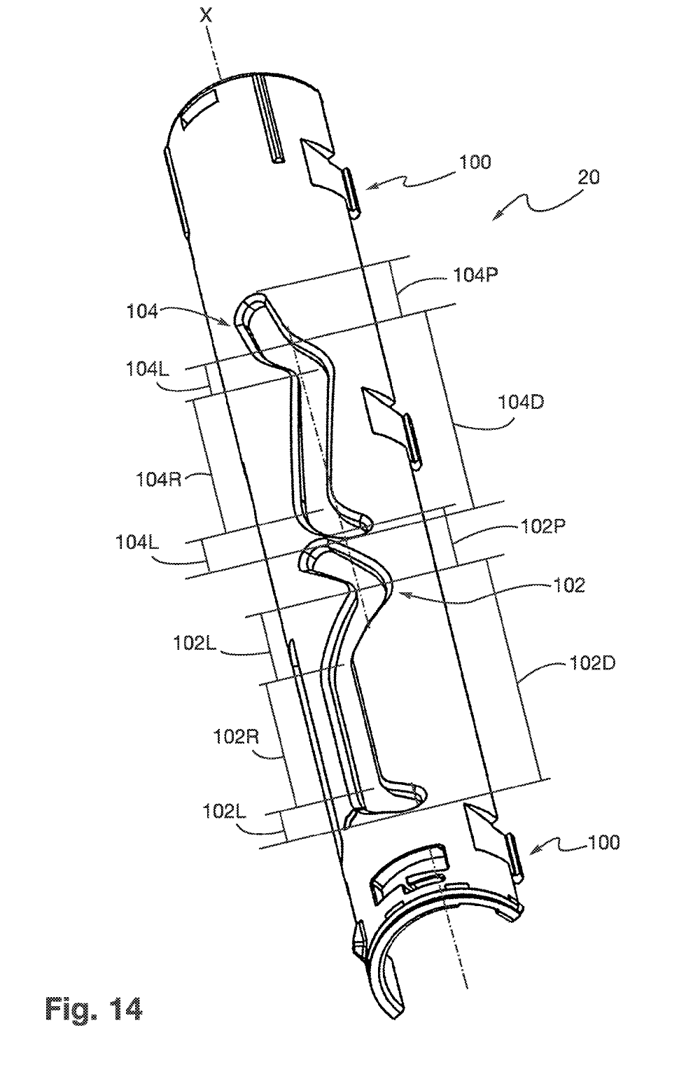

There will be described below, with reference to FIG. 14, a positioning controlling member 20 suitable for an injection device according to a second embodiment of the invention. In this FIG. 14 elements analogous to elements from the preceding figures are designated by identical references.

In this embodiment, the needle control member 21 and the piston control member 22 are both deceleration members (21,22). The needle control cam path 102 and the piston control cam path 104 are therefore deceleration cam paths and the needle control cam 46 and the piston control cam 106 are deceleration cams.

In this embodiment, the distal parts 102D and 104D, or injection parts, of the two deceleration cam paths 102 and 104 include a so-called fast section 102R and 104R interleaved between first and second so-called slow sections 102L and 104L. The fast sections 102R and 104R and the first and second slow sections 102L and 104L have different inclinations relative to the injection axis X such that the axial speed of the needle control member 21 and the piston control member 22 is lower when their deceleration cams 46 and 106 travel over the slow sections 102L and 104L than when they travel over the fast sections 102R and 104R. The choice of a greater inclination for a section enables reduction of the axial speed of the needle control member 21 and the piston control member 22 when the deceleration cam 46 or 106 travels over this section. In fact, greater rotation of one of the deceleration members 21 or 22 is then necessary to travel the same axial distance. This greater rotation causes more friction in various parts of the mechanism and therefore limits the speed of the needle control member 21 and the piston control member 22.

The deceleration cam paths 102 and 104 have sections 102R and 102L or 104R and 104L inclined relative to the injection axis X in opposite senses. These changes of inclination sense enable use of shorter lengths in some sections, notably the slow sections 102L and 104L, without the angle traveled being too great and does not trigger uncoupling of the syringe body 26 and the needle control member 21 or of the piston rod 19 and the piston control member 22.

The more distal two slow sections 102L and 104L of the deceleration cam paths 102 and 104 coincide with the distal end of the distal parts 102D and 104D of these deceleration cam paths 102 and 104. These two sections therefore impose a high deceleration at the end of the injection. During this deceleration the injection flow rate is virtually zero. The injected product therefore has the time to diffuse before the withdrawal of the injection needle 32 which occurs when the injection cam 106 reaches the distal end of the piston control cam path 104.

The more proximal two slow sections 102L and 104L of the deceleration cam paths 102 and 104 coincide with the proximal ends of the distal parts 102D and 104D of these deceleration cam paths 102 and 104. These two sections therefore impose a low speed at the start of injection. This low speed enables a more constant flow rate during the injection by preventing the flow rate from being higher at the start of the injection because of the greater compression of the injection spring 24.

It is therefore seen that the needle control cam path 102 includes four distinct sections, three sections 102L and 102R are located on the distal part 102D of the needle control cam path 102 while the fourth one is located on its proximal part 102P. Note that the section carried by the proximal part 102P has an inclination relative to the injection axis X greater than that of the fast section 102R. This inclination is chosen so that the axial speed of the needle control member 21 and the piston control 22 is lower when the needle control cam 46 of the needle control member 21 travels over the proximal section 102P of the needle control cam path 102 than when this needle control cam 46 travels over the fast section 102R.

It is also seen that the piston control cam path 104 includes four distinct sections, three sections 104L and 104R are located on the distal part 104D of the piston control cam path 104 while the fourth is located on its proximal part 104P. Note that the section carried by the proximal part 104P has an inclination close to that of the fast section 104R.

During the injection phase, the piston control cam 106 will therefore travel successively over the slow section 104L, the fast section 104R and the slow section 104L, leading to an injection speed that is successively slow, then fast then slow again.

Alternatively, the various fast and slow sections are not carried by the two deceleration cam paths 102 and 104; for example, only one of the needle control cam path 102 or the piston control path 104 of the positioning controlling member 20 comprises sections the inclinations of which are different.

According to another alternative or combined variant, two slow sections 102L or 104L carried by the same deceleration cam path 102 or 104 can have different inclinations.

According to a further alternate or combined variant, the piston control and needle control cam paths join to form a single cam path.

The main steps of assembling the injection device 10 are described next.

Overall, the assembly of the injection device 10 includes four main steps:
a step of assembling a distal subassembly comprising the syringe support 14,
a step of inserting the injection syringe 12 into the syringe support 14 from its proximal end 14P,
a step of inserting the preassembled positioning ring 50 with the piston rod 19 on the proximal end 14P of the syringe support 14, preferably positioned relative to one another in the axial direction thanks to the axial positioning means 94,
a step of assembling with the distal subassembly a proximal subassembly, mounted from above on the piston rod 19.

A first subassembly, termed the distal subassembly, is assembled first. A first step of this comprises nesting the end sleeve 16 with the withdrawal member 18. This step is followed by a step of clipping the syringe support 14 to the end sleeve 16 by nesting radial projections 52 of the syringe support 14 in proximal windows 80 of the end sleeve 16. The protection springs are threaded over guide rods of the syringe support 14 and the end sleeve 16 and held in place by clipping together the two parts.

Moreover, a second subassembly, termed the proximal subassembly, is assembled. For this, the positioning controlling member 20, the needle control member 21, the piston control member 22, the retaining ring 23, the injection spring 24 and the exterior casing 25 are assembled. To be more precise, the injection spring 24 is first assembled onto the proximal end of the piston control member 22. These elements are then placed in one of the two half-shells of the positioning controlling member 20, whilst also adding the needle control member 21 in the position coupled to the piston control member 22. The second half-shell of the positioning controlling member 20 is then fixed to the first half-shell, after which the exterior casing 25 is threaded around these elements, assembly being completed by clipping together the distal end of the exterior casing 25 and that of the positioning controlling member 20, this clipping nevertheless allowing the possibility of axial sliding, enabling the positioning controlling member 20 to move back relative to the exterior casing 25.

A third subassembly, termed the intermediate subassembly, is moreover assembled. This intermediate subassembly comprises the piston rod 19 fitted with the positioning ring 50.

The injection syringe 12, fitted with its protection cap 42 and the piston 40, moreover forms another separate element.

The injection device 10 is assembled by inserting the injection syringe 12 in the distal subassembly, by inserting the latter in the syringe support 14 from its proximal end. Once the injection syringe 12 and the distal subassembly have been assembled, the intermediate subassembly is mounted in the proximal end of the syringe body 26, by cooperation of the positioning ring 50 with the syringe support 14. The proximal subassembly is then fitted from the top of the piston rod 19, in particular by inserting the locking pin 107 of the needle control member 21 in a guide rail provided in the syringe support 14, and everything is locked by rotating the withdrawal member 18 relative to the exterior casing 25, in particular generating rotation of the locking pin 107 so that the external shoulder of the syringe support 14 is immobilized between the locking pin 107 and a guide rib.

Clearly this assembly is particularly easy, without requiring complex tools.

The invention is not limited to the embodiments described and other embodiments will be clearly apparent to the person skilled in the art. Among the multiple variants that can be envisaged, it will be noted that it would in particular be possible to screw the piston rod onto the piston. In this case, the positioning ring is assembled in the retracted position on the piston rod, after which the piston rod is screwed in and the positioning ring is then caused to slide so as to key it to the syringe support and activate the axial positioning means.

Moreover, it will be noted that, although there is described an injection device 10 configured to assure at the same time automatic insertion of the injection needle 32 in the body of the patient and automatic injection of the liquid product once the needle is inserted, it is possible to provide a deceleration member as described above on an injection device assuring only automatic injection of the product and the injection needle of which is configured to be inserted manually in the body of the patient.

1. A liquid product injection device, intended to receive an injection syringe comprising a syringe body, carrying an injection needle, and a piston mounted sliding axially in the syringe body in the distal direction when an injection is performed, the injection device comprising a piston control member intended to cooperate axially, because of the effect of an elastic force, with a piston rod, kinematically connected to the piston to cause it to slide, characterized in that the injection device further comprises at least one deceleration member kinematically connected in its movement in translation along an injection axis X to the piston control member, the deceleration member being mounted movable in a positioning controlling member with which it cooperates via a contact between a deceleration cam and a deceleration cam path, the deceleration cam and the deceleration cam path being one carried by the deceleration member and the other by the positioning controlling member, the deceleration cam path comprising an injection part such that when the deceleration cam travels over this injection part, the piston control member causes the piston to slide, the injection part of the deceleration cam path including at least two sections including a fast section and a slow section, with different inclinations, such that the axial speed of the piston control member, when the deceleration cam travels over the slow section, is less than the axial speed of this piston control member when the deceleration cam travels over the fast section, in that the deceleration cam is carried by the deceleration member and the deceleration cam path is carried by the positioning control member and in that the liquid product injection device comprises two deceleration members namely the piston control member and the needle control member.

2. The injection device according to claim 1, wherein the deceleration cam path includes at least two slow sections.

3. The injection device according to claim 2, wherein the two slow sections have different inclinations such that the axial speed of the piston control member, when the deceleration cam travels over a first slow section, is less than the axial speed of this piston control member when the deceleration cam travels over a second slow section.

4. The injection device according to claim 1, wherein the deceleration cam path includes three sections with different inclinations forming a fast section interleaved between first and second slow sections, so that the axial speed of the piston control member is successively slow, fast and slow when the deceleration cam travels over the slow, fast and slow sections.

5. The injection device according to claim 1, wherein the slow section of the injection part of the deceleration cam path coincides with a distal end or a proximal end of the injection part of this deceleration cam path, or with each of the distal and proximal ends.

6. The injection device according to claim 1, wherein the deceleration cam path includes at least two sections with different inclinations such that the angular movement of the deceleration cam traveling over them evolves successively in opposite rotation directions.

7. The injection device according to claim 1, wherein the deceleration member and the piston control member are one and the same.

8. The injection device according to claim 1, comprising a needle control member intended to cooperate axially, because of the effect of an elastic force with a syringe support, kinematically connected to the syringe body.

9. The injection device according to claim 8, wherein the deceleration member and the needle control member are one and the same.
